(19) United States
(12) Patent Application Publication
(10) Pub. No.: US 2026/0074035 A1
(43) Pub. Date: Mar. 12, 2026

(54) METHODS AND SYSTEMS FOR IMPROVING AN EFFECTIVENESS OF REFLECTION BY AN INDIVIDUAL MEMBER

(71) Applicant: Sequelae, Inc., Minneapolis, MN (US)

(72) Inventors: James LaBelle, Encinitas, CA (US); Benjamin C. Wiegand, Yardley, PA (US); Dominic Scheck, St. Anthony, MN (US); Kiersten Clements, Murray, UT (US); Mark Briesacher, Park City, UT (US); Steven J. Catani, Athens, GA (US)

(73) Assignee: Sequelae, Inc., Minneapolis, MN (US)

(21) Appl. No.: 19/321,862

(22) Filed: Sep. 8, 2025

Related U.S. Application Data
(60) Provisional application No. 63/691,455, filed on Sep. 6, 2024.

(51) Int. Cl. G16H 10/40; G16H 10/20; G16H 10/65; G16H 40/67

(52) U.S. Cl. CPC G16H 10/40; G16H 10/20; G16H 10/65; G16H 40/67

(57) ABSTRACT

Methods and systems for improving an effectiveness of reflection are provided. A digital communication network (DCN) is provided for a plurality of members of a population to interact with each other and the DCN. A communication from the DCN is transmitted to an individual member of the plurality of members and the communication comprises information about an availability of a biometric test to the individual member, and a request for the individual member to reflect on a lifestyle condition of the individual member and what the biometric test may reveal about the lifestyle condition. A response to the request is received from the individual member and the biometric test is provided to the individual member. A result of the biometric test is then reported to the individual member.

### CROSS-REFERENCE TO RELATED APPLICATIONS

This application claims the benefit of U.S. Application No. 63/691,455, filed on Sep. 6, 2024, which is incorporated herein by reference in its entirety.

### BACKGROUND

Various embodiments relate generally to improving an effectiveness of reflection by an individual member of a population and more specifically, to improving the effectiveness of reflection using a digital communications network (DCN).

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

People often take actions—whether simple, hard, good, or bad, etc.—that impact their health. Regardless, such actions have consequences that can accumulate over time. However, rarely do people associate such actions with consequences. This may be due to, for example, the lack of data to see the consequence (or the data is available in a way that does not connect to the action). Other times, they may not think about the connection between the action and the consequence. Reflecting on their conditions can help people associate actions with their consequences; thus, it is desirable for people to reflect on their actions and potential consequences.

Further, computers have changed the way people interact. Digital networks, which may include the use of social media, allow individuals to interact with others online and connect many people with a community. The online community can be utilized to help build positive behaviors and encourage people to make improvements in their lives. Computers can also provide a way for people to record their actions and review them later as well as to enable a person to reflect on their activities. Reflecting takes conscience effort and is often emotionally draining and cognitively draining. Thus, a person may find it easier to not reflect on such actions.

Thus, what is needed is a way to incentivize reflection in an individual member of their own lifestyle condition(s) prior to and after, for example, biological testing.

### SUMMARY

Example aspects of the present disclosure include:

A method according to at least one embodiment of the present disclosure comprises providing a digital communication network (DCN) for a plurality of members of a population to interact with each other and the DCN; transmitting a communication from the DCN to an individual member of the plurality of members, the communication comprising: information about an availability of a biometric test to the individual member, and a request for the individual member to reflect on a lifestyle condition of the individual member and what the biometric test may reveal about the lifestyle condition; receiving, by the DCN, a response to the request from the individual member; providing, by the DCN, the biometric test to the individual member; and reporting, by the DCN, a result of the biometric test to the individual member.

Any of the aspects herein, wherein the results of the biometric test are received from a biometric lab.

Any of the aspects herein, wherein the method is used to at least one of manage progression of a chronic medical condition in the individual member or improve a health and wellbeing of the individual member.

Any of the aspects herein, wherein the communication is a first communication, the request is a first request, and the response is a first response, and wherein the method further comprises: transmitting a second communication from the DCN to the individual member, the second communication including at least one of a second request for the individual member to reflect on the result of the biometric test or a follow-up question based in part on the result of the biometric test; and receiving a second response to the second request from the individual member, the second response including a reflection from the individual member.

Any of the aspects herein, wherein the communication is a first communication and the response is a first response, and wherein the method further comprises: transmitting a second communication from the DCN to the individual member, the second communication including a follow-up question based on the first response to the request; and receiving a second response to the follow-up question.

Any of the aspects herein, further comprising: providing at least one of a communications template or a digital tool to the individual member to facilitate the request to reflect.

Any of the aspects herein, wherein the digital tool comprises a chat bot.

Any of the aspects herein, further comprising: sharing the response to the request to at least one other member of the plurality of members.

Any of the aspects herein, wherein the biometric test derives biometric data from one of: saliva, blood, breath, urine, stool, a wearable device, a psychometric survey, and results of the individual member playing a game.

Any of the aspects herein, wherein the response to the request as at least one of: a textual communication, a verbal communication, or a visual communication.

Any of the aspects herein, wherein reporting the result of the biometric test includes sending a copy of the response to the request to the individual member.

A system according to at least one embodiment of the present disclosure comprises a computer processor; a data repository in communication with the computer processor and storing: a communication comprising information about an availability of a biometric test and a request for an individual member to reflect on a lifestyle condition of the individual member and what the biometric test may reveal about the lifestyle condition, a response to the request, and a result of the biometric test; a digital communications network which, when executed by the computer processor, provides a network for members of a population to interact with each other; and a server controller which, when executed by the computer processor: transmits the communication to the individual member; receives a response to the request from the individual member; provides the biometric test to the individual member; and reports the result of the biometric test to the individual member.

Any of the aspects herein, wherein the results of the biometric test are received from a biometric lab.

Any of the aspects herein, wherein the system is used to at least one of manage progression of a chronic medical condition in the individual member or improve a health and wellbeing of the individual member.

Any of the aspects herein, wherein the communication is a first communication, the request is a first request, and the response is a first response, and wherein the server controller further: transmits a second communication from the DCN to the individual member, the second communication including at least one of a second request for the individual member to reflect on the result of the biometric test or a follow-up question based in part on the result of the biometric test; and receives a second response to the second request from the individual member, the second response including a reflection from the individual member.

Any of the aspects herein, wherein the communication is a first communication and the response is a first response, and wherein the server controller further: transmits a second communication from the DCN to the individual member, the second communication including a follow-up question based on the first response to the request; and receives a second response to the follow-up question.

Any of the aspects herein, wherein the server controller further: provides at least one of a communications template or a digital tool to the individual member to facilitate the request to reflect.

Any of the aspects herein, wherein the digital tool comprises a chat bot.

Any of the aspects herein, wherein the server controller further: shares the response to the request to at least one other member of the plurality of members.

Any of the aspects herein, wherein the biometric test derives biometric data from one of: saliva, blood, breath, urine, stool, a wearable device, a psychometric survey, and results of the individual member playing a game.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE DRAWINGS

Aspects of the described embodiments are more evident in the following description, when read in conjunction with the attached Figures.

FIG. 1 shows a simplified block diagram of devices, in accordance with one or more embodiments.

FIG. 2 is a logic flow diagram that illustrates the operation of a method, in accordance with one or more embodiments.

FIG. 3A shows an example of a computing system, in accordance with one or more embodiments.

FIG. 3B shows an example of a network, in accordance with one or more embodiments.

### DETAILED DESCRIPTION

Reflection is normally considered after an event. A person can observe a change and reflect on the cause of that change. This reflection helps understand why a change, good or bad, had occurred and can teach the person how to achieve better results. Often these changes are observed through objective data, such as, a biometric sample, results tracked through a smartwatch, or some other means that bring the change to life. When observations are made, reflection before the result is known can provide a way to make a post-observation reflection more powerful. Various embodiments use a digital communication network (DCN) to encourage an individual to also reflect before a biometric test result is known in order to improve the effectiveness of reflection to assist in treating a medical condition.

Reflections are an important part of a person's experience. They allow the person to turn observations about the connection between their actions and the consequences of those actions into useable insight. Digital health applications typically provide people access to data about their health or health-related actions and often allow them to record observations about how they feel. This can cause reflection but in a minimal way.

Reflection may be considered as part of the change process—a person does or observes something, and then reflects on the action or observation and the consequences. This process implies some degree of planning, either in adding reflection to a planned action, or scheduling periods to reflect (these can be formal as in professional counseling). Unfortunately, much of life is unplanned and unobserved.

Thus, embodiments of the present disclosure provide for improving an effectiveness of reflection in an individual member by requesting reflection in the individual member prior to and after biometric testing. By requesting reflection prior to and after the biometric testing, the individual member may be more likely to more deeply reflect on their own actions and lifestyle that may have attributed to their test results that they were unaware of in their reflection prior to receiving their test results.

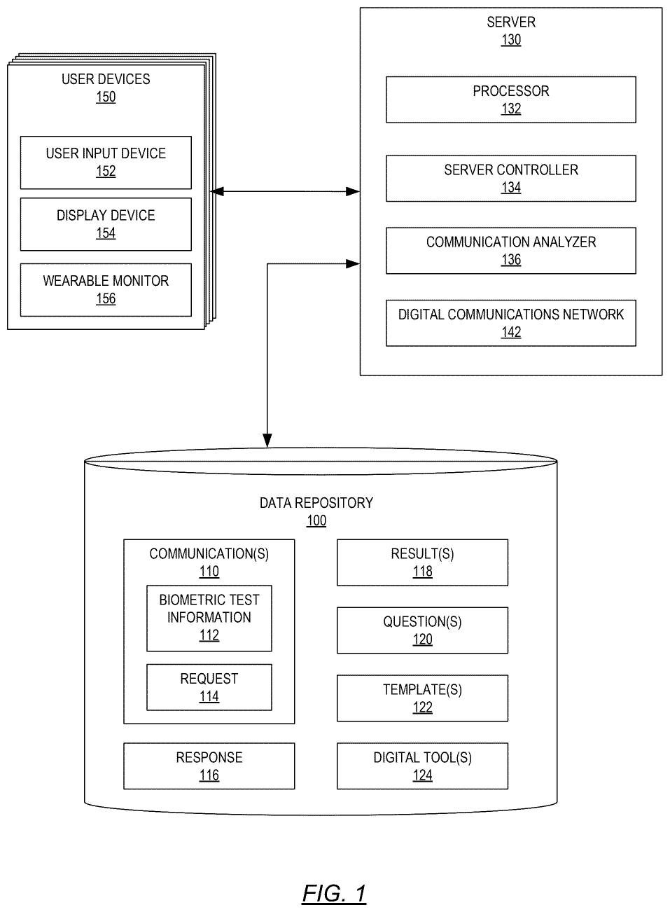

The system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

The data repository (100) stores communications (110). The communications (110) can be communications from members in a digital communications network (DCN) (142) or from the DCN (142). The communications (110) may be, for example, text-based, image-based, multimedia communications, audio communications, or any combinations thereof. The communications (110) may be between members, communications in a forum, communications with the DCN (142), or communications generated by the DCN (142). The communications (110) can be one-to-one communications, one-to-many communications, one-to-system communications, system-to-one communications, and system-to-many communications.

The communication (110) can include biometric test information (112) and a request (114). The biometric test information (112) can include information such as instructions on where and how to obtain a biometric test. For example, the biometric test information (112) may include instructions for requesting the biometric test to be mailed to the individual member or information about a clinic location closest to the individual member. The biometric test information (112) may also include instructions on how to self-administer the biometric test or how to book an appointment for a health care provider to administer the biometric test. The biometric test can derive biometric data from one of: saliva, blood, breath, urine, stool, a wearable device, a psychometric survey, and/or results of the individual member playing a game.

The request (114) includes a request for the individual member to reflect on what the biometric test may reveal about their own health or a lifestyle condition of the individual member. The request (114) can also include a subsequent or second request for the individual member to reflect on results (118) from the biometric test. The request (114) may include one request, two requests, or more than two requests.

The data repository (100) also stores responses (116). The responses (116) are responses (116) to the request (114) in the communication (110) and/or responses (116) to questions (120) from the individual member. The responses (116) may include, for example, a yes/no answer, text, and/or media (e.g., audio, visual, images, videos, etc.). The response (116) may also be a verbal communication, a verbal communication converted to a textual communication, a visual communication, or a graphical communication.

The response (116) may also include a reflection from the individual member. The reflection may be a reflection on what the biometric test may reveal about their own health or a lifestyle condition of the individual member and/or a reflection on the results (118) from the biometric test.

The data repository (100) also stores results (118). The results (118) may be received from, for example, a biometric testing lab or facility. The results (118) may include data such as biological markers, which are a measurable characteristic of a biological state or condition or what is happening in an organism or a cell. The biological markers are measured and evaluated using a blood, urine, stool, saliva, breath, or soft tissue sample from a user or member of a population or from heart-rate meters, accelerometer samples, body temperature as well as other signals derived from wearables. The biological markers can also be derived from psychometric instruments and EMA-derived data.

The data repository (100) also stores questions (120). The questions (120) are questions directed to the individual member and may include yes/no questions or open-ended questions. The questions (120) may be designed so as to illicit reflection from the individual member about what a biological test may reveal about their health or lifestyle condition and/or the results (118) from the biological test. The questions (120) can include one question, two questions, or more than two questions.

The data repository (100) also stores templates (122). The templates (122) may be templates (122) to induce reflection in the individual member. The templates (122) may include, for example, prompts and/or instructions for the individual member to respond to or fill out. For example, the template (122) may include instructions to fill out the individual member's lifestyle condition and to answer questions (120) such as “what do you think contributed to your lifestyle condition?” and “what do you think your biometric test will reflect about you?”.

The data repository (100) also stores digital tools (124). The digital tools (124) can be, for example, a chat or conversation bot. The chat bot may also be configured to provide prompts and/or instructions for the individual member to respond to as well, but in the form of a real-time conversation with the chat bot. The chat bot may also respond to the individual member's answers and responses and may provide follow-up or additional prompts and/or questions. The chat bot will be described in more detail with respect to the DCN (142) below.

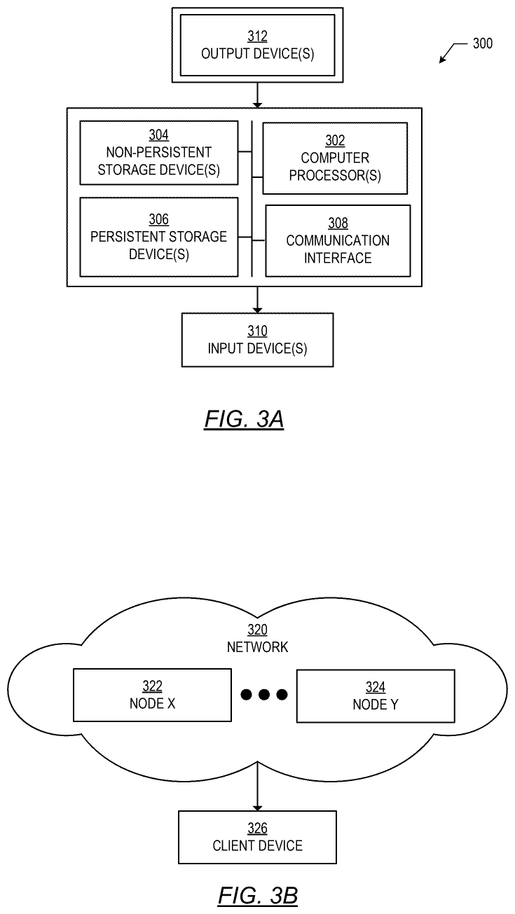

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such as a server controller (134) and a communication analyzer (136). An example of a computer system and network that may form the server (130) is described with respect to FIG. 3A and FIG. 3B.

The server (130) also includes a computer processor (132). The computer processor (132) is one or more hardware or virtual processors which may execute computer readable program code that defines one or more applications, such as the server controller (134) and the communication analyzer (136). An example of the computer processor (132) is described with respect to the computer processor(s) (302) of FIG. 3A.

The server (130) also includes a communication analyzer (136). The communication analyzer (136) is software or application specific hardware which, when executed by the computer processor (132), receives response(s) (116) as input and outputs communications (110) which may include questions (120) tailored to the responses (116). The questions (120) may be used to encourage the individual member to provide reflection on their results (118) or may be used to clarify the individual member's responses (116). The communication analyzer (136) can also generate communications (110) for the DCN (142) to send to members of the population regarding, for example, the individual member's responses (116).

The server (130) also may include the server controller (134). The server controller (134) is software or application specific hardware which, when executed by the computer processor (132), controls and coordinates operation of the software or application specific hardware described herein. Thus, the sever controller (138) may control and coordinate execution of the DCN (142) and the communication analyzer (136).

The server (130) also includes the DCN (142). The DCN (142) is a network through which members of a population can interact with each other, or with a system supported by the DCN (142). The DCN (142) can also interact with the members of the population and, for example, transmit communications (110) to the individual member to induce reflection in the individual member prior to and after a biometric test. The DCN (142) can also interact with the members of the population to inform them of the existence of biometric testing available to the members.

In some embodiments, the DCN (142) may be, for example, an artificial intelligence bot configured to derive generated communications from analysis of existing communications (110) in the DCN (142) or biometrics provided to the DCN (142). Biometric data can be used in many instances such as the provisioning and commissioning of healthcare. For example, biometric data can be used to diagnose diabetes, prescribe hypertension medication with a patient's blood pressure exceeds a certain value, prescribe cancer drugs, etc. In another example, cancer drug can be available for reimbursement if a certain genetic signature is present in the tumor. The decision tree and other logic behind these relationships considers many things including what is known about the variance in the intervention's efficacy, therapeutic index, the accuracy and precision in the biometric data's measurement, as well as the cost-benefit ratio of the intervention compared with other interventional options.

The biometric data, as well as reflections from the individual member, can be used in a “feedback” decision tree where, for example, an intervention can be prescribed or recommended to an individual member based on the biometric data and/or the reflections. Then the biometric data or additional reflections can be obtained to study the effects or results of the intervention.

In some embodiments, the DCN (142) uses a conversation bot to help patients with seeking help for medical conditions. The conversation bot is an artificial intelligence (AI) tool that can review community discussions on the DCN (142) to, for example, identify key issues that may be a concern for a patient. The bot can ask questions of the individual member so that they can reflect on their situation and/or better appreciate the consequences of any actions. This can help identify conditions, reactions, etc. The bot can also review community discussions to identify possible questions that should be asked of a doctor to help a patient with the preliminary work and setting them up to get the key information for proper medical intervention and treatment.

In some embodiments, the conversation bot may use a visual or graphical interface, for example, a rendered avatar, or a plot or image from the bot or sharing one with the bot. The DCN (142) can send a visual representation showing that person's data from a wearable, their interaction with the community, their consistency in talking with the bot, etc. The conversation bot may then ask questions like “What do you notice” or “Is anything here surprising”in order to induce a reflection from the person.

The conversation bot can analyze data from various sources, in part, to formulate its communications with the individual member. These sources include data from i) the DCN (142), ii) the ongoing conversation with the individual member, and iii) previous conversations with the individual member. The conversation bot can also use temporal and situational cues to formulate its communication next with the member. When analyzing the data, the conversation bot may quote sections of the conversations for use with communications between the individual member and/or the conversation bot may identify patterns in the discussions. When formulating the communications, the conversation bot can analyze exchanges in the DCN (142) from other members. These communications can be limited to specifically target individuals, such as those deemed to be close contacts to the individual member or members that are considered influencers.

One-to-one communications with the conversation bot can be triggered and informed by one-to-many communications in the DCN (142), and conversations with the conversation bot can (a) refer individuals to the relevant communications in the DCN (142), (b) suggest posts to the DCN (142), and (c) make reference to ideas contained in posts. The two ways to interact can behave in a synergistic way and allow the conversation bot to include a “human in the loop”in a surprisingly scalable way.

During the communication between the individual member and the conversation bot, the DCN (142) can analyze the individual member's responses. Based on the input from the individual member, the DCN (142) may identify an adverse health condition or a lifestyle condition for the individual member. The conversation bot may then gather more information regarding the adverse health condition, for example, to develop more details to confirm the adverse health condition. The communication may be based on related experiences from another member of the population. The conversation bot may quote content from a post by that other member.

The synergy between the conversation bot and the DCN (142) can facilitate reflection on a topic. Alternatively, this can be seen to be reversed where the act of facilitating reflection synergizes with usage of the bot in the DCN (142).

Using analysis of the individual member's communications in the DCN (142), the conversation bot can suggest a reflection on a topic in a one-to-one communication. The suggestion may be made as part of a question to the individual member. The conversation bot may then focus its conversations with that individual member on that topic for a period of time (e.g., a number of exchanges, a few minutes, a few days, etc.). The conversation bot can incorporate excerpts of communication on the DCN (142) in its responses. The excerpts of communications may include post titles, post topics, quotes from the body of the communication, and/or post contributors. Also, the excerpts could be summaries of communications on the DCN (142). These summaries may be created automatically by the DCN (142) and/or are based on a topic, a member's communications, or a time period.

The bot may prompt the individual member to reflect on various classes of action. The action may be active or passive. The action may also be one that was contemplated even if not undertaken.

Two or more conversation bots, each with a different personality or a different role (e.g. facilitate reflections, find useful communications, say good morning and inspire a positive start to a day, handle routine network tasks, provide custom communication summaries), may be provided by the DCN (142). The family of bots could each use conversations between the other bots and the user on the DCN (142) to formulate their responses. When working with an individual member, the bots may be selected based on the personality of the bot that is expected to work best with that user. The decision may be based on past dealings with the individual member and/or through analysis of that individual member's communication on the DCN (142). For example, when attempting to induce a reflection, the bot may use a first personality, then switch to a second personality when helping draft a post regarding that reflection. Accordingly, various embodiments can be used to provide a method to improve the communications of the conversation bot through using authentic experiences of the individual member in the communication.

The system shown in FIG. 1 also may include one or more user devices (150). The user devices (150) may be considered remote or local. A remote user device is a device operated by a third-party (e.g., an end user of a chatbot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

In any case, the user devices (150) are computing systems (e.g., the computing system (300) shown in FIG. 3A) that communicate with the server (130). The user devices (150) may include a wearable monitor (156) and be configured to send stress indicator data (110) to the server (130). In an alternative embodiment, a separate wearable device may be in communication with the user device (150), such as a smart watch, or blood pressure monitor. The user devices (150) may also include a user input device (152) and/or a display device (154).

In contrast, a local user device is a device operated under the control of the organization that controls the other components of the system of FIG. 1. Thus, a local user device may be considered part of the system of FIG. 1.

While FIG. 1 shows a configuration of components, other configurations may be used without departing from the scope of one or more embodiments. For example, various components may be combined to create a single component. As another example, the functionality performed by a single component may be performed by two or more components.

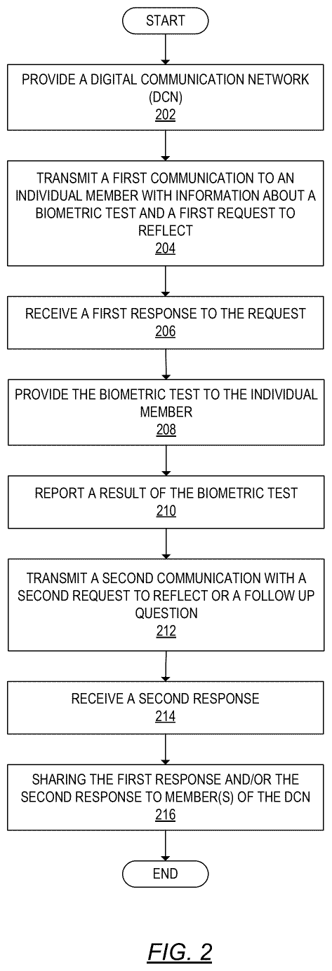

FIG. 2 is a logic flow diagram that illustrates a method, and a result of execution of computer program instructions, in accordance with various embodiments. The method can be used to improve an effectiveness of reflection in an individual member. The method can also, in other instances, be used in part to facilitate or accomplish at least one of: manage the health care cost of the population; reduce the health care risk in the population; and/or slow the progression of an adverse health condition or a lifestyle driven disease. The lifestyle driven disease may be, for example, a metabolic disease, an auto-immune disease, or a cardiovascular disease.

At Block 202, a digital communication network (DCN) is provided. The DCN may be the same as or similar to the DCN (142) and provides a network for members of a population to interact with each other or with the DCN.

At Block 204, a step of transmitting a first communication to an individual member with information about a biometric test and a first request to reflect is provided. The first communication is the same as or similar to the communication (110), the biometric test information is the same as or similar to the biometric test information (112), and the first request is the same as or similar to the request (114). The first communication may be transmitted to the individual member at a specific time at which the individual member is most likely to respond. For example, the first communication may be transmitted to the individual member after the individual member has exercised and may be open to learning more about their own health and lifestyle.

As previously described, the communication may include the request for the individual member to reflect on what the biometric test may reveal about their own health or a lifestyle condition of the individual member. In some embodiments, a conversation bot may provide some basic information regarding the biometric test and then guide the individual member's reflections through a series of questions about their lifestyle condition, their experiences, and possible expectations. The request may also include a template such as the template (122), digital tools such as the digital tools (124), and/or questions such as the questions (120) to aid the individual member in reflecting.

At Block 206, a step of receiving a first response to the request is provided. The first response may be the same as or similar to the response (116) and may include the individual member's reflection of their own lifestyle condition and what the biometric test may reveal about their lifestyle condition. The first response may be received by the DCN.

At Block 208, a step of providing the biometric test to the individual member is provided. In response to receiving the first response, the biometric test may be provided to the individual member. Providing the biometric test to the individual member may include automatically mailing the biometric test to the individual member and/or enabling the individual member to schedule a biometric test at a testing facility or clinic.

At Block 210, a step of reporting a result of the biometric test is provided. The result may be the same as or similar to the result (118) and may be received by the DCN from, for example, a testing lab that processes data from the biometric test. In some embodiments, a copy of the first response may be reported with the results to the individual member to enable the individual member to reflect on their initial reflection and the results of the biometric test.

At Block 212, a step of transmitting a second communication with a second request to reflect or one or more follow up questions is provided. The Bock 212 may be the same as or similar to the Block 204. Additionally, the follow up questions, follow up templates, or continued conversation with a conversation bot may be generated based on an analysis of the individual member's results and/or first response. Such follow up questions, templates, or conversations with the conversation bot may be generated by, for example, a communication analyzer such as the communication analyzer (136) which analyzes the individual member's reflection in the first response and the individual member's results.

It will be appreciated that communications including templates, questions, and requests for reflection may be provided throughout the process. In other words, the questions may be provided at different times, such as when ordering the test, when taking the test, immediately before receiving the results, immediately after receiving the results, and/or sometime after receiving the results.

At Block 214, a step of receiving a second response is provided. The Bock 214 may be the same as or similar to the Block 206. By requesting that the individual member reflect prior to and after the biometric test, their reflection may be more effective and may lead to the individual member to adjusting their own lifestyle or adopting new, healthy lifestyles. Their reflection may be more effective due to having the individual member identify their own lifestyle conditions, how the biometric test may reflect such lifestyle conditions, and reflecting on the results of the biometric test relative to their lifestyle condition. For example, the biometric test may reveal that the individual member is negatively affected by their lifestyle condition more than the individual member expected. Such misalignment in their expected result versus the actual result may lead the individual member to adopt habits to improve their lifestyle condition. Further, such reflections may create greater awareness of lifestyle changes and medial options. The individual member's reflections can also be used to evaluate a lifestyle (or behavior) change. Such induced reflections may be used to help identify the individual member's condition, for example, to evaluate whether a lifestyle change is working.

At Block 216, a step of sharing the first response and/or the second response to other members of the DCN is provided. The first response and/or the second response may be shared anonymously to the members of the DCN or may be shared by the individual member themselves. The first response and/or the second response may also be shared to a subset of members that may have been identified as having the same lifestyle condition as the individual member or may benefit from the individual member's experience. By sharing such responses to other members of the DCN, those members may be incentivized to also reflect on their lifestyle condition and to obtain biometric testing as value can be placed on those members of a community that have relevant experience. As more members participate in reflecting and biometric testing and potentially change their lifestyle or adopt healthy habits in response to their reflections, the overall health of the population increases.

One or more embodiments may be implemented on a computing system specifically designed to achieve an improved technological result. When implemented in a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 3A, the computing system (300) may include one or more computer processor(s) (302), non-persistent storage device(s) (304), persistent storage device(s) (306), a communication interface (308) (e.g., Bluetooth interface, infrared interface, network interface, optical interface, etc.), and numerous other elements and functionalities that implement the features and elements of the disclosure. The computer processor(s) (302) may be an integrated circuit for processing instructions. The computer processor(s) (302) may be one or more cores, or micro-cores, of a processor. The computer processor(s) (302) includes one or more processors. The computer processor(s) (302) may include a central processing unit (CPU), a graphics processing unit (GPU), a tensor processing unit (TPU), combinations thereof, etc.

The input device(s) (310) may include a touchscreen, keyboard, mouse, microphone, touchpad, electronic pen, or any other type of input device. The input device(s) (310) may receive inputs from a user that are responsive to data and messages presented by the output device(s) (312). The inputs may include text input, audio input, video input, etc., which may be processed and transmitted by the computing system (300) in accordance with one or more embodiments. The communication interface (308) may include an integrated circuit for connecting the computing system (300) to a network (not shown) (e.g., a local area network (LAN), a wide area network (WAN) such as the Internet, mobile network, or any other type of network) or to another device, such as another computing device, and combinations thereof.

Further, the output device(s) (312) may include a display device, a printer, external storage, or any other output device. One or more of the output device(s) (312) may be the same or different from the input device(s) (310). The input device(s) (310) and output device(s) (312) may be locally or remotely connected to the computer processor(s) (302). Many different types of computing systems exist, and the aforementioned input device(s) (310) and output device(s) (312) may take other forms. The output device(s) (312) may display data and messages that are transmitted and received by the computing system (300). The data and messages may include text, audio, video, etc., and include the data and messages described above in the other figures of the disclosure.

Software instructions in the form of computer readable program code to perform embodiments may be stored, in whole or in part, temporarily or permanently, on a non-transitory computer readable medium such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (302), is configured to perform one or more embodiments, which may include transmitting, receiving, presenting, and displaying data and messages described in the other figures of the disclosure.

The computing system (300) in FIG. 3A may be connected to, or be a part of, a network. For example, as shown in FIG. 3B, the network (320) may include multiple nodes (e.g., node X (322) and node Y (324), as well as extant intervening nodes between node X (322) and node Y (324)). Each node may correspond to a computing system, such as the computing system shown in FIG. 3A, or a group of nodes combined may correspond to the computing system shown in FIG. 3A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (300) may be located at a remote location and connected to the other elements over a network.

The nodes (e.g., node X (322) and node Y (324)) in the network (320) may be configured to provide services for a client device (326). The services may include receiving requests and transmitting responses to the client device (326). For example, the nodes may be part of a cloud computing system. The client device (326) may be a computing system, such as the computing system shown in FIG. 3A. Further, the client device (326) may include or perform all or a portion of one or more embodiments.

The computing system of FIG. 3A may include functionality to present data (including raw data, processed data, and combinations thereof) such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user. Furthermore, the GUI may present data directly to the user, e.g., data presented as actual data values through text, or rendered by the computing device into a visual representation of the data, such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer-readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

What is claimed is:

1. A method comprising:
providing a digital communication network (DCN) for a plurality of members of a population to interact with each other and the DCN;
transmitting a communication from the DCN to an individual member of the plurality of members, the communication comprising:
information about an availability of a biometric test to the individual member, and
a request for the individual member to reflect on a lifestyle condition of the individual member and what the biometric test may reveal about the lifestyle condition;
receiving, by the DCN, a response to the request from the individual member;
providing, by the DCN, the biometric test to the individual member; and
reporting, by the DCN, a result of the biometric test to the individual member.

2. The method of claim 1, wherein the results of the biometric test are received from a biometric lab.

3. The method of claim 1, wherein the method is used to at least one of manage progression of a chronic medical condition in the individual member or improve a health and wellbeing of the individual member.

4. The method of claim 1, wherein the communication is a first communication, the request is a first request, and the response is a first response, and wherein the method further comprises:
transmitting a second communication from the DCN to the individual member, the second communication including at least one of a second request for the individual member to reflect on the result of the biometric test or a follow-up question based in part on the result of the biometric test; and
receiving a second response to the second request from the individual member, the second response including a reflection from the individual member.

5. The method of claim 1, wherein the communication is a first communication and the response is a first response, and wherein the method further comprises:
transmitting a second communication from the DCN to the individual member, the second communication including a follow-up question based on the first response to the request; and
receiving a second response to the follow-up question.

6. The method of claim 1, further comprising:
providing at least one of a communications template or a digital tool to the individual member to facilitate the request to reflect.

7. The method of claim 6, wherein the digital tool comprises a chat bot.

8. The method of claim 1, further comprising:
sharing the response to the request to at least one other member of the plurality of members.

9. The method of claim 1, wherein the biometric test derives biometric data from one of:
saliva, blood, breath, urine, stool, a wearable device, a psychometric survey, and results of the individual member playing a game.

10. The method of claim 1, wherein the response to the request as at least one of: a textual communication, a verbal communication, or a visual communication.

11. The method of claim 1, wherein reporting the result of the biometric test includes sending a copy of the response to the request to the individual member.

12. A system comprising:
a computer processor;
a data repository in communication with the computer processor and storing:
a communication comprising information about an availability of a biometric test and a request for an individual member to reflect on a lifestyle condition of the individual member and what the biometric test may reveal about the lifestyle condition,
a response to the request, and
a result of the biometric test;
a digital communications network which, when executed by the computer processor, provides a network for members of a population to interact with each other; and
a server controller which, when executed by the computer processor:
transmits the communication to the individual member;
receives a response to the request from the individual member;
provides the biometric test to the individual member; and
reports the result of the biometric test to the individual member.

13. The system of claim 12, wherein the results of the biometric test are received from a biometric lab.

14. The system of claim 12, wherein the system is used to at least one of manage progression of a chronic medical condition in the individual member or improve a health and wellbeing of the individual member.

15. The system of claim 12, wherein the communication is a first communication, the request is a first request, and the response is a first response, and wherein the server controller further:
transmits a second communication from the DCN to the individual member, the second communication including at least one of a second request for the individual member to reflect on the result of the biometric test or a follow-up question based in part on the result of the biometric test; and
receives a second response to the second request from the individual member, the second response including a reflection from the individual member.

16. The system of claim 12, wherein the communication is a first communication and the response is a first response, and wherein the server controller further:
transmits a second communication from the DCN to the individual member, the second communication including a follow-up question based on the first response to the request; and
receives a second response to the follow-up question.

17. The system of claim 12, wherein the server controller further:
provides at least one of a communications template or a digital tool to the individual member to facilitate the request to reflect.

18. The system of claim 17, wherein the digital tool comprises a chat bot.

19. The system of claim 12, wherein the server controller further:
shares the response to the request to at least one other member of the plurality of members.

20. The system of claim 12, wherein the biometric test derives biometric data from one of: saliva, blood, breath, urine, stool, a wearable device, a psychometric survey, and results of the individual member playing a game.
